(19) United States
(12) Patent Application Publication
(10) Pub. No.: US 2006/0178543 A1
(43) Pub. Date: Aug. 10, 2006

(54) Foamed sheet containing a styrenic copolymer

(76) Inventors: Steven Michael Krupinski, Pittsburgh, PA (US); Charlie Ravagni, Lowell, MA (US); Thomas J. Styranec, Poland, OH (US); Richard Delaney JR., Lunenburg, MA (US); Don Thomas Harris, Cranberry Township, PA (US)

Correspondence Address: Gary F. Matz;NOVA Chemicals Inc., 400 Frankfort Rd., Monaca, PA, 15061, US

(21) Appl. No.: 11/143,267

(22) Filed: Jun. 2, 2005

Related U.S. Application Data
(60) Provisional application No. 60/650,992, filed on Feb. 8, 2005.

(51) Int. Cl. B29C 44/00; B32B 3/26; C07C 2/72; B29C 39/00

(52) U.S. Cl. 585/428; 428/304.4; 264/415

(57) ABSTRACT

A foamed sheet containing a polymer composition including a polymer formed by polymerizing a mixture including 40% to 90% of styrenic monomers; 5% to 45% of maleate-type monomers; 0.1% to 25% by weight of elastomeric polymers with Mn greater than 6,000; and 0.1% to 10% by weight of low molecular weight polymers that include one or more monomers according to the formula CH₂═CR³R², where R³ is H or a C₁-C₃ alkyl group and R² is a C₁-C₂₂ linear, branched or cyclic alkyl or alkenyl groups. The low molecular weight polymer has Mn of from 400 to 6,000 and can optionally include functional groups. The foamed sheet is made by injecting blowing agents into the polymer melt composition; mixing the blowing agent with the polymer composition; and extruding the mixture to provide a foamed sheet. The foamed sheets can be thermoformed into containers suitable for use in microwave heating of food.

### REFERENCE TO RELATED APPLICATION

This application claims the benefit of priority of U.S. Provisional Application Ser. No. 60/650,992 filed Feb. 8, 2005 and entitled “Foamed Sheet Containing A Styrenic Copolymer,” which is herein incorporated by reference in its entirety.

### BACKGROUND OF THE INVENTION

1. Field of the Invention

The present invention is directed to foamed thermoplastic sheets containing styrenic copolymers and articles formed from such foamed sheets.

2. Description of the Prior Art

It is known to produce various shaped articles from foamed and unfoamed thermoplastic materials such as polystyrene sheet or impact modified polystyrene sheet (i.e. high impact polystyrene sheet) by thermoforming methods. Many such articles are containers used for packaged foods.

U.S. Pat. No. 5,106,696 to Chundury et al. discloses and claims a thermoformable multi-layer structure for packaging materials and foods. A polymer composition for a first layer of the structure includes: (A) 49% to 90% by weight of a polyolefin, i.e. polypropylene, polybutene; (B) 10% to 30% by weight of a copolymer of styrene and maleic anhydride; (C) 2% to 20% by weight of a compatabillizing agent, i.e., a starblock, diblock or mixtures thereof of a copolymer of styrene and butadiene; (D) 0 to 5% by weight of a triblock copolymer of styrene and butadiene; and (E) 20% by weight of talc. The second layer of the structure is made of polypropylene.

It is known to improve the environmental stress crack resistance (ESCR) of high impact polystyrene (HIPS) and other impact modified styrenic polymers, such as acrylonitrile-butadiene-styrene plastic (ABS) and methyl methacrylate-butadiene-styrene plastics (MBS), with the addition of polybutene. U.S. Pat. No. 5,543,461 discloses a rubber modified graft thermoplastic composition containing: 1) 99 to 96% by weight of a rubber modified thermoplastic including: (a) 4 to 15 weight % rubbery substrate, preferably polybutadiene, that is distributed throughout a matrix of the superstrate polymer in particles having a number average particle size from 6 to 12 microns and (b) 96 to 85% by weight of a superstrate polymer; and 2) 1 to 4% by weight of polybutene having a number average molecular weight from 900 to 2000. Such thermoplastics are used, for example, in housewares, which are typically subjected to chemicals that tend to cause environmental stress cracking (ESC), such as cleaners and in some cases, fatty or oily food.

U.S. Pat. No. 5,543,461 discussed in the preceding paragraph discloses in the background section that the thermoplastic having the best ESCR is Chevron's HIPS grade 6755. This Chevron product contains 2 to 3 weight % of polybutene and has a dispersed rubbery phase with a volume average particle diameter between 4 and 4.5 microns. This Chevron product relates to high impact polystyrene (HIPS) with ESCR properties and not to a rubber modified styrene/maleic anhydride copolymer.

U.S. Pat. No. 3,919,354 discloses an improved styrene/maleic anhydride/diene rubber composition suitable for extrusion and molding and having a high heat distortion temperature and desired impact resistance. The process for the preparation of the polymer involves modifying a styrene-maleic anhydride copolymer with diene rubber by polymerizing the styrene monomer and the anhydride in the presence of the rubber. More particularly, the process involves providing a styrene having rubber dissolved therein; agitating the styrene/rubber mixture and initiating free radical polymerization thereof; adding to the agitated mixture the maleic anhydride at a rate substantially less than the rate of polymerization of the styrene monomer; and polymerizing the styrene monomer and the maleic anhydride. The polymer contains rubber particles ranging from 0.02 to 30 microns dispersed throughout a matrix of polymer of the styrene monomer and the anhydride with at least a major portion of the rubber particles containing occlusions of the polymerized styrene monomer and maleic anhydride. This patent teaches that the polymers are suited for extrusion into sheet or film, which are then employed for thermoforming into containers, packages and the like. Alternately the polymers can be injection molded into a wide variety of components such as dinnerware and heatable frozen food containers.

However, polymers as those disclosed in the above U.S. Pat. No. 3,919,354 are generally brittle, and therefore, capable of breaking even though these polymers have the thermal properties to withstand temperatures above 210° F., which temperature is generally used in heating food in a microwave oven.

Further, containers used for heating food or liquids in a microwave oven made from known foamed materials often have an altered or distorted shape after heating, break and or leak, especially during removal from the microwave oven.

There is a need in the art for articles, such as containers that are suitable for packaged foods that can withstand the temperatures needed for heating foods in a microwave oven without the container breaking, especially upon removal of the container out of a microwave oven.

### SUMMARY OF THE INVENTION

The present invention provides a foamed sheet that contains a polymer composition containing a polymer formed by polymerizing a mixture that includes:
about 40% to about 90% by weight of one or more styrenic monomers;
about 5% to about 45% by weight of one or more maleate-type monomers;
about 0.1% to about 25% by weight of one or more elastomeric polymers having a number average molecular weight greater than 6,000; and
about 0.1% to about 10% by weight of one or more low molecular weight polymers comprising one or more monomers according to the formula CH₂═CR³R², wherein R³ is H or a C₁-C₃ alkyl group and R² is a C₁-C₂₂ linear, branched or cyclic alkyl or alkenyl group, wherein the low molecular weight polymer has a number average molecular weight of from 400 to 6,000 and can optionally include one or more functional groups selected from the group consisting of hydroxyl, amine, epoxy, carboxylic acid, carboxylic acid esters, and carboxylic acid anhydrides.

The present invention is also directed to a method of making a foamed sheet that includes providing the above described polymer composition in polymer melt form; injecting into the polymer melt composition from 2 to 15 weight % based on the polymer composition of one or more blowing agents; mixing the blowing agent with the polymer composition; and extruding the mixture of blowing agent and polymer composition to provide a foamed sheet.

The present invention further provides articles produced from the above-described foamed sheets as well as containers suitable for use in microwave heating of food formed from the above-described foamed sheets.

The present invention additionally provides a container suitable for use in microwave heating of food formed by thermoforming a foamed sheet that includes a polymer composition containing a copolymer formed by polymerizing a mixture containing:
about 40% to about 90% by weight of one or more styrenic monomers; and
about 5% to about 45% by weight of one or more maleate-type monomers;
   and combining the copolymer with
about 0.1% to about 25% by weight of one or more elastomeric polymers having a number average molecular weight of greater than 6,000; and

optionally up to about 10% by weight of one or more low molecular weight polymers comprising one or more monomers according to the formula CH₂═CR³R², wherein R³ is H or a C₁-C₃ alkyl group and R² is a C₁-C₂₂ linear, branched or cyclic alkyl or alkenyl group, wherein the low molecular weight polymer has a number average molecular weight of from 400 to 6,000 and can optionally include one or more functional groups selected from the group consisting of hydroxyl, amine, epoxy, carboxylic acid, carboxylic acid esters, and carboxylic acid anhydrides.

### DESCRIPTION OF THE DRAWINGS

FIG. 1 is a photomicrograph of a cross-section of a comparative polystyrene foamed sheet;

FIG. 2 is a photomicrograph of a cross-section of a foamed sheet according the invention;

FIG. 3 is a photomicrograph of a cross-section of a sheet without a blowing agent;

FIG. 4 is a photomicrograph of a cross-section of a foamed sheet according the invention;

FIG. 5 is a photomicrograph of a cross-section of a foamed sheet according the invention;

FIG. 6 is a photomicrograph of a cross-section of a foamed sheet according the invention; and

FIG. 7 is a photomicrograph of a cross-section of a foamed sheet according the invention.

### DETAILED DESCRIPTION OF THE INVENTION

Other than in the operating examples or where otherwise indicated, all numbers or expressions referring to quantities of ingredients, reaction conditions, etc. used in the specification and claims are to be understood as modified in all instances by the term “about.” Accordingly, unless indicated to the contrary, the numerical parameters set forth in the following specification and attached claims are approximations that can vary depending upon the desired properties, which the present invention desires to obtain. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical values, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, the terms “(meth)acrylic” and “(meth)acrylate” are meant to include both acrylic and methacrylic acid derivatives, such as the corresponding alkyl esters often referred to as acrylates and (meth)acrylates, which the term “(meth)acrylate” is meant to encompass.

As used herein, the term “polymer” is meant to encompass, without limitation, homopolymers, copolymers and graft copolymers.

As used herein, the term “high impact polystyrene” refers to rubber modified polystyrene as is known in the art. Also, “crystal polystyrene” refers to polystyrene that does not contain other polymers, a non-limiting example being rubber.

As used herein, “rubber-modified copolymers of styrene and maleic anhydride and/or C₁-C₁₂ linear, branched or cyclic alkyl (meth)acrylates” refer to polymer compositions that include copolymers of styrene and maleic anhydride and/or C₁-C₁₂ linear, branched or cyclic alkyl (meth)acrylates and a rubber and that are not encompassed by the description of the present polymer composition and in particular that do not include the low molecular weight polymer as described herein.

Unless otherwise specified, all molecular weight values are determined using gel permeation chromatography (GPC) using appropriate polystyrene standards. Unless otherwise indicated, the molecular weight values indicated herein are weight average molecular weights (Mw).

The present invention is directed to a foamed sheet. As used herein, the term “foamed sheet” refers to a sheet having a length corresponding to the extruding direction (machine direction) of an extruder, a width corresponding to the direction perpendicular (traverse direction) to the extruding direction and a thickness. The foamed sheet is characterized as containing a thermoplastic material having a cellular structure. The cellular structure results from the process described herein below.

The foamed sheet in the present invention contains a polymer composition that includes a copolymer formed by polymerizing a polymerization mixture containing one or more styrenic monomers, one or more maleate-type monomers, and combining the copolymer with one or more elastomeric polymers, and optionally one or more low molecular weight polymers.

The styrenic monomers are present in the polymerization mixture and/or the formed copolymer at a level of at least 40%, in some cases at least 45% and in other cases at least 50% and can be present at up to 90%, in some cases up to 85%, in other cases up to 80%, and in some situations up to 75% by weight based on the polymerization mixture and/or the formed copolymer. The styrenic monomers can be present in the polymerization mixture and/or the formed copolymer at any level or can range between any of the values recited above.

Any suitable styrenic monomer can be used in the invention. Suitable styrenic monomers are those that provide the desirable properties in the present foamed sheet as described below. Non-limiting examples of suitable styrenic monomers include styrene, p-methyl styrene, α-methyl styrene, tertiary butyl styrene, dimethyl styrene, nuclear brominated or chlorinated derivatives thereof and combinations thereof.

The maleate-type monomers are present in the polymerization mixture and/or the formed copolymer at a level of at least 5%, in some cases at least 10% and in other cases at least 15% and can be present at up to 45%, in some cases up to 40%, in other cases up to 35%, and in some situations up to 30% by weight based on the polymerization mixture and/or the formed copolymer. The maleate-type monomers can be present in the polymerization mixture and/or the formed copolymer at any level or can range between any of the values recited above.

Any suitable maleate-type monomer can be used in the invention. Suitable maleate-type monomers are those that provide the desirable properties in the present foamed sheet as described below and include anhydrides, carboxylic acids and alkyl esters of maleate-type monomers, which include, but are not limited to maleic acid, fumaric acid and itaconic acid. Specific non-limiting examples of suitable maleate-type monomers include maleic anhydride, maleic acid, fumaric acid, C₁-C₁₂ linear, branched or cyclic alkyl esters of maleic acid, C₁-C₁₂ linear, branched or cyclic alkyl esters of fumaric acid, itaconic acid, C₁-C₁₂ linear, branched or cyclic alkyl esters of itaconic acid, and itaconic anhydride.

The elastomeric polymers are combined with the copolymer and, in a particular embodiment of the invention, are present in the polymerization mixture at a level of at least 0.1%, in some cases at least 0.5%, in other cases at least 1%, and in some instances at least 2% and can be present at up to 25%, in some cases up to 20%, in other cases up to 15%, and in some situations up to 10% by weight based on the weight of the polymer composition. The elastomeric polymers can be present at any level or can range between any of the values recited above.

Any suitable elastomeric polymer can be used in the invention. In some embodiments of the invention, combinations of elastomeric polymers are used to achieve desired properties. Suitable elastomeric polymers are those that provide the desirable properties in the present foamed sheet as described below and are desirably capable of resuming their shape after being deformed.

In an embodiment of the invention, the elastomeric polymers include, but are not limited to homopolymers of butadiene or isoprene or other conjugated diene, and random, block, AB diblock, or ABA triblock copolymers of a conjugated diene (non-limiting examples being butadiene and/or isoprene) with a styrenic monomer as defined above and/or acrylonitrile.

In a particular embodiment of the invention, the elastomeric polymers include one or more block copolymers selected from diblock and triblock copolymers of styrene-butadiene, styrene-butadiene-styrene, styrene-isoprene, styrene-isoprene-styrene, partially hydrogenated styrene-isoprene-styrene and combinations thereof.

As used herein, butadiene refers to 1,3-butadiene and when polymerized, to repeat units that take on the 1,4-cis, 1,4-trans and 1,2-vinyl forms of the resulting repeat units along a polymer chain.

In an embodiment of the invention, the elastomeric polymer has a number average molecular weight (Mn) greater than 6,000, in some cases greater than 8,000, and in other cases greater than 10,000 and a weight average molecular weight (Mw) of at least 25,000 in some cases not less than about 50,000, and in other cases not less than about 75,000 and the Mw can be up to 500,000, in some cases up to 400,000 and in other cases up to 300,000. The weight average molecular weight of the elastomeric polymer can be any value or can range between any of the values recited above.

Non-limiting examples of suitable block copolymers that can be used in the invention include the STEREON® block copolymers available from the Firestone Tire and Rubber Company, Akron Ohio; the ASAPRENE™ block copolymers available from Asahi Kasei Chemicals Corporation, Tokyo Japan; the KRATON® block copolymers available from Kraton Polymers, Houston, Tex.; and the VECTOR® block copolymers available from Dexco Polymers LP, Houston, Tex.

The low molecular weight polymers are optionally combined with the copolymer and, in a particular embodiment of the invention, are present in the polymerization mixture at a level of at least 0.1%, in some cases at least 0.25%, in other cases at least 0.5%, and in some instances at least 1% and can be present at up to 10%, in some cases up to 7.5%, and in other cases up to 5% by weight based on the polymer composition. The low molecular weight polymers can be present at any level or can range between any of the values recited above.

Any suitable low molecular weight polymer can be used in the invention. In some embodiments of the invention, combinations of low molecular weight polymers are used to achieve desired properties.

Suitable low molecular weight polymers desirably include repeat units resulting from the polymerization of one or more monomers according to the formula CH₂═CR³R², where R³ is H or a C₁-C₃ alkyl group, in some cases a C₁-C₂ alkyl group, in some instances a C₂-C₃ alkyl group, and in other cases methyl and R² is a C₁-C₂₂, in some cases C₁-C₁₈, in other cases C₁-C₁₂, in some circumstances C₂-C₂₂, in other circumstances C₂-C₁₈, in some situations C₂-C₁₂, in other situations C₂-C₆ and in some instances C₁-C₆ linear, branched or cyclic alkyl or alkenyl group, including conjugated dienes.

In a particular embodiment of the invention, when R² is methyl, R³ is not methyl and when R³ is methyl R² is not methyl.

In an embodiment of the invention, the low molecular weight polymers include repeat units resulting from the polymerization of one or more monomers selected from 1 -butene, isobutylene, 2-butene, isoprene, butadiene, diisobutylene, 1-pentene, 2-pentene, 1-hexene, 2-hexene, 3-hexene, 1,3-hexadiene, 2,4-hexadiene, isoprenol, ethylene, propylene and combinations thereof.

In an embodiment of the invention, the low molecular weight polymers include one or more functional groups selected from hydroxyl, amine, epoxy, carboxylic acid, C₁-C₁₂ in some cases C₁-C₆ linear, branched or cyclic alkyl carboxylic acid esters, and carboxylic acid anhydride.

The low molecular weight polymers of the present invention can have a number average molecular weight of at least 400, in some cases at least 500, and in other cases at least 750 and up to 6,000, in some circumstances up to 5,000, in some cases up to 4,000, in other cases up to 3,000 and in some instances up to 2,000. The molecular weight can be determined using gel permeation chromatography (GPC) using polystyrene standards. The molecular weight of the low molecular weight polymers can be any value or can range between any of the values recited above.

In a particular embodiment of the invention, the low molecular weight polymers include polybutenes. Suitable polybutenes that can be used in the invention include, but are not limited to the INDOPOL® and PANALANE® products available from AMOCO Chemical Company, Chicago, Ill.

In another particular embodiment of the invention, the low molecular weight polymers include polybutadienes. Suitable polybutadienes that can be used as the low molecular weight polymer of the invention include, but are not limited to the KRASOL® products available from Kaucuk, a.s., Czech Republic.

The polymer composition can be prepared by polymerizing the polymerization mixture in a suitable reactor under free radical polymerization conditions. The low molecular weight polymer can be added to a styrenic monomer/maleate-type monomer/elastomeric polymer feed, or can be added to or in the polymerization reactor vessel, or can be added to the partially polymerized syrup after it exits the reactor and enters the devolatilizer. It is also envisioned that the low molecular weight polymer can be compounded, i.e. mixed into the copolymer after the copolymer has exited a devolatilizer, via an extruder, e.g. a twin-screw extruder, either in line or off line as a separate operation after the rubber-modified SMA copolymer has been pelletized.

The term “devolatilizer” and the term “devolatilizing system” as used herein are meant to include all shapes and forms of devolatilizers including an extruder and/or a falling strand flash devolatilizer. The term “devolatilizing” and the term “devolatilizing step” as used herein are meant to refer to a process, which can include an extruder and/or a falling strand flash devolatilizer.

In an embodiment of the invention, a low molecular weight polymer is added to the reacting mixture of elastomeric polymer, styrenic monomer and maleate-type monomer before the devolatilization step to improve toughness, elongation, and heat distortion resistance properties of the polymer composition, foamed sheets and articles made according to the invention. This polymer composition can be used in applications where prior art resins have proven to be too brittle and/or the heat distortion resistance is inadequate. For example, and as discussed hereinabove, if containers for packaged foods made from the rubber-modified styrenic/maleic anhydride resins of the prior art are heated in microwave ovens at temperatures higher than 210° F., the containers generally break when they are taken out of the oven. The foamed sheet of the present invention can now be used in making these types of containers without the containers breaking under normal usage.

The reason for the improvements in the polymer composition of the invention is not clear, and the inventors do not wish to be bound to any particular theory. However, it is believed that the addition of low molecular weight polymer to the styrenic monomer/maleate-type monomer/elastomeric polymer combination before devolatilizing distributes the low molecular weight polymer such that it enhances the properties of the elastomeric polymer component. In other words, it is believed that the low molecular weight polymer gravitates toward, surrounds and migrates into the elastomeric polymer and not the forming styrenic/maleate-type monomer component in view of the high polarity of the styrenic/maleate-type monomer matrix. In contrast, it is theorized that the low molecular weight polymer used particularly in accordance with the teachings of U.S. Pat. No. 5,543,461 is distributed in the matrix along with the polystyrene and the rubber component.

U.S. Pat. No. 5,543,461 teaches that the rubber-modified thermoplastic composition can be rubber-modified styrene/maleic anhydride copolymer and polybutene. However, the Examples in the '461 patent only illustrate high impact polystyrene (HIPS) and improvements in ESCR, and both the Examples and the teachings of this '461 patent are silent regarding enhancement in toughness or producing foamed sheets as described herein.

Thus, U.S. Pat. No. 5,543,461 teaches that the polybutene ranges in amounts from 1 to 4% by weight and the rubber particle size ranges from 6 to 12 microns. The inventors have found that the rubber particle size used in the polymeric composition of the invention is desirably less than 6 microns in order to provide the desired properties.

The polymer composition of the invention can be prepared via polymerization techniques or compounding techniques, both of which are known to those skilled in the art.

It has been found that the addition of the low molecular weight polymer to the reactor or to the syrup exiting the reactor and prior to it entering the devolatilizer can provide an even higher degree of improvement in toughness, elongation, and heat distortion resistance properties compared to the addition of the polybutene in a compounding technique which entails the polybutene being added to the polymer in an extruder after the devolatilizer and the pelletizer or after the devolatilizer but before the pelletizer, more about which will be discussed in greater detail herein below.

The polymerization techniques used in polymerizing the components of the polymer composition of the invention can be solution, mass, bulk, suspension, or emulsion polymerization. In an embodiment of the invention, bulk polymerization methods are used.

The polymer composition can be prepared by reacting styrenic monomers, maleate-type monomers, and elastomeric polymers in a suitable reactor under free radical polymerization conditions and adding the low molecular weight polymer to the reactive mixture. Desirably the maleate-type monomers are added to the styrenic monomers and the elastomeric polymer continuously at about the rate of reaction to a stirred reactor to form a polymer composition having a uniform maleate-type monomer level.

Polymerization of the polymerization mixture can be accomplished by thermal polymerization generally between 50° C. and 200° C.; in some cases between 70° C. and 150° C.; and in other cases between 80° C. and 140° C. Alternately free-radical generating initiators can be used.

Non-limiting examples of free-radical initiators that can be used include benzoyl peroxide, 2,4-dichlorobenzoyl peroxide, di-tert-butyl peroxide, tert-butyl peroxybenzoate, dicumyl peroxide, cumene hydroperoxide, diisopropylbenzene hydroperoxide, diisopropyl peroxydicarbonate, tert-butyl perisobutyrate, tert-butyl peroxyisopropylcarbonate, tert-butyl peroxypivalate, methyl ethyl ketone peroxide, stearoyl peroxide, tert-butyl hydroperoxide, lauroyl peroxide, azo-bis-isobutyronitrile and mixtures thereof.

Generally, the initiator is included in the range of 0.001 to 1.0% by weight, and in some cases on the order of 0.005 to 0.5% by weight of the polymerization mixture, depending upon the monomers and the desired polymerization cycle.

In an embodiment of the invention, the polymer composition is prepared by solution or bulk polymerization in the presence of from 0.01 to 0.1 weight % based on the mixture of a tetra functional peroxide initiator of the formula: 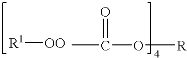
where R¹ is selected from C₄₋₆ t-alkyl radicals and R is a neopentyl group, in the absence of a cross linking agent. In a particular embodiment of the invention, the tetrafunctional initiator is selected from the group consisting of tetrakis-(t-amylperoxycarbonyloxymethyl) methane, and tetrakis-(t-butylperoxycarbonyloxymethyl) methane.

In some cases, the required total amount of initiator is added simultaneously with the feedstock when the feedstock is introduced into the reactor.

Customary additives known in the art, such as stabilizers, antioxidants, lubricants, fillers, pigments, plasticizers, etc., can be added to the polymerization mixture. If desired, small amounts of antioxidants, such as alkylated phenols, e.g., 2,6-di-tert-butyl-p-cresol, phosphates such as trinonyl phenyl phosphite and mixtures containing tri (mono and dinonyl phenyl) phosphates, can be included in the feed stream. Such materials, in general, can be added at any stage during the polymerization process.

A polymerization reactor that can be used in producing the polymer composition of the invention is similar to that disclosed in the aforesaid U.S. Pat. Nos. 2,769,804 and 2,989,517, the teachings of which patents are incorporated in their entirety herein by reference. These configurations are adapted for the production, in a continuous manner, of solid, moldable polymers and copolymers of vinylidene compounds, particularly that of monovinyl aromatic compounds, i.e. styrene. Of these two arrangements, that of U.S. Pat. No. 2,769,804 is particularly desirable. Further, the polymer composition of the present invention can be prepared as disclosed in U.S. Application Publication 2005/0020756.

In general, the arrangement of U.S. Pat. No. 2,769,804 provides for an inlet or inlets for the monomers or feedstock connected to the polymerization reactor vessel. The reactor vessel is surrounded by a jacket, which has an inlet and an outlet for passage of a temperature control fluid through the jacket, and a mechanical stirrer. A valve line leads from a lower section of the vessel and connects with a devolatilizer, which can be any of the devices known in the art for the continuous vaporization and removal of volatile components from the formed resin exiting the vessel. For example, the devolatilizer can be a vacuum chamber through which thin streams of heated resin material pass, or a set of rolls for milling the heated polymer inside of a vacuum chamber, etc. The reactor is provided with usual means such as a gear pump for discharging the heat-plastified polymer from the reactor to the devolatilizer. A vapor line leads from the devolatilizer to a condenser, which condenses the vapors and effects the return of the recovered volatiles, e.g. monomeric material, typically in liquid condition as a recycle stream.

In general, the arrangement for producing the polymer composition will include at least three apparatuses. These are a polymerization reactor vessel assembly that can include one or more reactor vessels, a devolatilizing system, and a pelletizer. As discussed hereinabove, some embodiments according to the invention utilize processes where the low molecular weight polymer is added to the polymer at one of three locations, i.e. to the reactor vessel; after the reactor vessel and prior to the devolatilizing system; or in a pelletizing extruder wherein compounding or mixing of the polybutene into the polymer occurs.

More particularly, a first method for preparing the polymer composition of the invention is to prepare a solution of the components, i.e. the low molecular weight polymer, maleate-type monomers, elastomeric polymer, and optionally an antioxidant and to dissolve this solution in the styrenic monomers which then is fed continuously to a polymerization reactor vessel that is equipped with a turbine agitator similar to that described in the preceding paragraph. The initiator can be added to the reactor vessel in a second stream. The reactor is stirred so that the contents are well mix and the temperature is maintained by the cooling fluid flowing in the reactor jacket. The exit stream is continuously fed into the devolatilizer (first extruder), and the final product is pelletized.

A second method involves adding the low molecular weight polymer and the styrenic monomer, maleate-type monomer, and elastomeric polymer feed separately to the polymerization reactor vessel and then polymerizing the feed in the presence of the low molecular weight polymer and the elastomeric polymer followed by devolatilizing the stream that exits the reactor vessel. The finished product can be pelletized after the devolatilizing system.

A third method involves forming a solution of maleate-type monomer and elastomeric polymer in styrenic monomer, continuously feeding this solution with the styrenic monomer into the polymerization reactor vessel to produce a partially polymerized styrenic syrup, and adding the low molecular weight polymer to the partially polymerized syrup as it exits the reactor vessel and prior to this syrup entering the devolatilizing system. The finished product can be pelletized after the devolatilizing system.

A fourth method involves forming a solution of maleate-type monomer and elastomeric polymer in styrenic monomer, continuously feeding the solution with the styrenic monomer into a polymerization reactor vessel to produce a partially polymerized styrenic syrup, devolatilizing the stream exiting the polymerization reactor vessel, and compounding or mixing the low molecular weight polymer into the polymer stream either in an in-line extruder followed by pelletizing or in a separate extrusion step after the rubber-modified styrenic monomer—maleate-type monomer copolymer has been pelletized.

A fifth method involves forming a copolymer of maleate-type monomer and styrenic monomer and subsequently compounding the elastomeric polymer and optionally low molecular weight polymer into the copolymer.

The polymerization generally occurs at a conversion of from 20 to 95%.

Typically, the polymerization process results in the styrenic and maleate-type monomers copolymerizing to form a continuous phase with the elastomeric polymer present in a dispersed phase. In an embodiment of the invention, at least some of the polymers in the continuous phase are grafted onto the elastomeric polymers in the dispersed phase.

In an embodiment of the invention, the dispersed phase is present as discrete particles dispersed within the continuous phase. Further to this embodiment, the volume average particle size of the dispersed particulate phase in the continuous phase is at least about 0.1 μm, in some cases at least 0.5 μm and in other cases at least 1 μm. Also, the volume average particle size of the dispersed phase in the continuous phase can be up to about 11 μm, in some cases up to 6 μm, in other cases up to 5.5 μm, in some instances up to 5 μm and in other instances up to 4 μm. The particle size of the dispersed phase in the continuous phase can be any value recited above and can range between any of the values recited above.

In another embodiment of the invention, the aspect ratio of the discrete particles is from at least about 1, in some cases at least about 1.5 and in other cases at least about 2 and can be up to about 5, in some cases up to about 4 and in other cases at least up to about 3. The aspect ratio of the dispersed discrete particles can be any value or range between any of the values recited above. As a non-limiting example, the aspect ratio can be measured by scanning electron microscopy or light scattering.

The average particle size and aspect ratio of the dispersed phase can be determined using low angle light scattering. As a non-limiting example, a Model LA-910 Laser Diffraction Particle Size Analyzer available from Horiba Ltd., Kyoto, Japan can be used. As a non-limiting example, a rubber-modified polystyrene sample can be dispersed in methyl ethyl ketone. The suspended rubber particles can then be placed in a glass cell and subjected to light scattering. The scattered light from the particles in the cell can be passed through a condenser lens and converted into electric signals by detectors located around the sample cell. As a non-limiting example, a He-Ne laser and/or a tungsten lamp can be used to supply light with a shorter wavelength. Particle size distribution can be calculated based on Mie scattering theory from the angular measurement of the scattered light.

The resulting copolymer from the above-described processes can have a weight average molecular weight (Mw, measured using GPC with polystyrene standards) of at least 20,000, in some cases at least 35,000 and in other cases at least 50,000. Also, the Mw of the resulting polymer can be up to 1,000,000, in some cases up to 750,000, and in other cases up to 500,000. The Mw of the resulting polymer can be any value or range between any of the values recited above.

The polymer composition according to the invention can be characterized as having a VICAT softening temperature of greater than 100° C., in some circumstances greater than 110° C., in other circumstances greater than 115° C., in some cases greater than 117.5° C., in other cases greater than 119° C., and in some instances greater than 120° C. and can be up to 135° C. in some cases 130° C. The VICAT softening temperature is determined according to ASTM-D1525. The VICAT softening temperature can be any value or range between any of the values recited above.

In order to form a foamed sheet, the above-described polymer composition is provided in polymer melt form, typically by heating the polymer composition above its melting temperature. A blowing agent is injected into the polymer melt composition, mixed into the polymer composition and the mixture of blowing agent and polymer composition is then extruded to form a foamed sheet.

In an embodiment of the invention, a compounded blend can be used that includes the present polymer composition and one or more other polymers. Suitable other polymers that can be blend compounded with the present polymer composition include, but are not limited to crystal polystyrene, high impact polystyrenes, polyphenylene oxide, copolymers of styrene and maleic anhydride and/or C₁-C₁₂ linear, branched or cyclic alkyl (meth)acrylates, rubber-modified copolymers of styrene and maleic anhydride and/or C₁-C₁₂ linear, branched or cyclic alkyl (meth)acrylates, polycarbonates, polyamides (such as the nylons), polyesters (such as polyethylene terephthalate, PET), polyolefins (such as polyethylene, polypropylene, and ethylene-propylene copolymers), polyvinylidene fluoride, acrylonitrile/(meth)acrylate copolymers, ethylene/vinyl acetate copolymers, ethylene vinyl alcohol copolymers, and combinations thereof.

When a compounded blend is used, the blend will typically include at least 10%, in some cases at least 25%, and in other cases at least 35% and up to 90%, in some cases up to 75%, and in other cases up to 65% by weight based on the blend of the present polymer composition. Also, the blend will typically include at least 10%, in some cases at least 25%, and in other cases at least 35% and up to 90%, in some cases up to 75%, and in other cases up to 65% by weight based on the blend of the other polymers. The amount of the present polymer composition and other polymers in the blend is determined based on the desired properties in the resulting foamed sheet and or formed article. The amount of the present polymer composition and other polymers in the blend can be any value or range between any of the values recited above.

Typically, the blowing agents are added to the polymer composition or blend at a level of at least 2%, in some cases at least 2.5%, in other cases at least 3%, and in some instances at least 4% and can be up to 15%, in some cases up to 12.5%, and in other cases up to 10% by weight based on the polymer composition. The amount of blowing agent used can be any value or can range between any of the values recited above.

Any suitable blowing agents can be used in the present invention so long as they expand and evaporate under extrusion conditions to form the desired cellular structure in the foamed sheet as discussed below.

Suitable blowing agents that can be used in the invention include, but are not limited to nitrogen, sulfur hexafluoride (SF₆), argon, carbon dioxide, 1,1,1,2-tetrafluoroethane (HFC-134a), 1,1,2,2-tetrafluoroethane (HFC-134), 1,1,1,3,3-pentafluoropropane, difluoromethane (HFC-32), 1,1 -difluoroethane (HFC-152a), pentafluoroethane (HFC-125), fluoroethane (HFC-161) and 1,1,1-trifluoroethane (HFC-143a), methane, ethane, propane, n-butane, isobutane, n-pentane, isopentane, cyclopentane, neopentane, hexane, azodicarbonamide, azodiisobutyro-nitrile, benzenesulfonylhydrazide, 4,4-oxybenzene sulfonyl-semicarbazide, p-toluene sulfonyl semi-carbazide, barium azodicarboxylate, N, N′-dimethyl-N, N′-dinitrosoterephthalamide, trihydrazino triazine, mixtures of citric acid and sodium bicarbonate, and combinations thereof.

The polymer composition or blend can be foamed using conventional extrusion foaming equipment. The extruder can be a back-to-back type or it can be a multizoned extruder having at least a first or primary zone to melt the polymer and inject blowing agent and a second extruder or zone.

As a non-limiting example, in the primary extruder or zone the polymer melt can be maintained at temperatures from about 425° F. to 450° F. (about 218 to 232° C.). Once the polymer is melted, blowing agent is injected into the melt at the end of the primary extruder or zone. In the primary extruder or zone there will be a high shear zone to promote thorough mixing of the blowing agent with the polymer melt. Such a zone can include a number of pin mixers.

The polymer melt containing dissolved or dispersed blowing agent can then be fed from the primary extruder to the secondary extruder or pass from a primary zone to a secondary zone within the extruder maintained, as a non-limiting example, at a melt temperature of 269° F. to 290° F. (about 132° C. to 143° C.). In the secondary extruder or zone the polymer melt and entrained blowing agent passes through the extruder barrel by the action of an auger screw having deep flights and exerting low shear upon the polymer melt. The polymer melt is cooled by means of cooling fluid, typically oil which circulates around the barrel of the extruder. Generally the melt is cooled to a temperature of from about 250° F. to about 290° F. (about 121° C. to 143° C.).

In an embodiment of the invention, the blowing agent can include from 30 to 95, in some cases from 70 to 95, and in other cases from 80 to 90 weight % of CO₂ and from 70 to 5, in some cases from 30 to 5, and in other cases from 20 to 10 weight % of one or more compounds selected from C₁-2 halogenated alkanes and C₄₋₆ alkanes. Suitable C₁₋₂ halogenated alkanes include the chloroflurocarbons (CFCs); hydrofluorocarbons (HFCs) and the hydrochlorofluorocarbons (HCFCs) such as trichlorofluoromethane (CFC-11); dichlorodifluoromethane (CFC-12); trichlorotrifluoroethane (CFC-113); dichlorotetrafluoroethane (CFC- 114); dichlorofluoromethane (CFC-21); chlorodifluoromethane (HCFC-22); difluoromethane (HFC-32); 2-chloro-1,1,1,2-tetrafluoroethane (HCFC-124); pentafluoroethane (HFC-125); 1,1,1,2-tetrafluoroethane (HCFC-124); 1,1-dichloro-1-fluoroethane (HCFC-141b); 1-chloro-1,1-difluoroethane (HCFC-142b); trifluoroethane (HFC-143a); 1,1-difluoroethane (HFC-152a); tetrafluoroethane (HFC-134a); and dichloromethane. However, due to environmental concerns it is desirable to use alkanes such as C₄₋₆ alkanes, which have not been halogenated such as butane, pentane, isopentane and hexane. The blowing agent system can be used in amounts from 2 to 15, in some cases from 2 to 10, and in other cases from about 3 to 8 weight % based on the weight of the polymer.

The pressure within the extruder should be sufficient to keep the blowing agent in the polymer melt. Typically, the pressures in the melt after the blowing system has been injected will be from about 1500 to 3500 psi, in some cases from about 2000 to about 2500 for CO₂. The CO₂ and the other blowing agent can be injected separately into the melt. If this is done, often the alkane and/or halogenated alkane will be injected upstream of the CO₂ as these types of blowing agents have a plasticizing effect on the polymer melt which can help the CO₂ incorporate into the melt. The alkane blowing agent and the CO₂ can also be mixed prior to injection into the extruder as is disclosed in U.S. Pat. No. 4,424,287.

To improve the cell size and/or distribution throughout the polymer small amounts of a nucleating agent can be incorporated into the polymer blend or solution. These agents can be physical agents such as talc or they can be agents which release small amounts of CO₂ such as citric acid and alkali or alkaline earth metal salts thereof and alkali or alkaline earth metal carbonates or bicarbonates. Such agents can be used in amounts from about 500 to 5,000 ppm, typically from about 500 to 2,500 ppm based on the polymer melt or blend.

Suitable nucleating agents that can be used in the invention include, but are not limited to calcium carbonate, barium stearate, calcium stearate, aluminum oxide, aluminum hydroxide, talc, clay, titanium dioxide, silica, diatomaceous earth, mixtures of citric acid and sodium bicarbonate, and combinations thereof. The nucleating agents are added to the polymer composition prior to extruding.

The polymer melt or blend can also contain conventional additives known in the art such as heat and light stabilizers (e.g. hindered phenols and phosphite or phosphonite stabilizers) typically in amounts of less than about 2 weight % based on the polymer blend or solution.

Other additives can be added to and/or compounded into the polymer composition for foamed sheets according to the invention. Further examples of suitable additives are softening agents; plasticizers, such as cumarone-indene resin, a terpene resin, and oils in an amount of about 2 parts by weight or less based on 100 parts by weight of the polymer; dyes, pigments; anti-blocking agents; slip agents; lubricants; coloring agents; antioxidants; ultraviolet light absorbers; fillers; anti-static agents; impact modifiers. Pigment can be white or any other color. The white pigment can be produced by the presence of titanium oxide, zinc oxide, magnesium oxide, cadmium oxide, zinc chloride, calcium carbonate, magnesium carbonate, etc., or any combination thereof in the amount of 0.1 to 20% in weight, depending on the white pigment to be used. The colored pigment can be produced by carbon black, phtalocianine blue, Congo red, titanium yellow or any other coloring agent known in the printing industry.

Examples of anti-blocking agents, slip agents or lubricants are silicone oils, liquid paraffin, synthetic paraffin, mineral oils, petrolatum, petroleum wax, polyethylene wax, hydrogenated polybutene, higher fatty acids and the metal salts thereof, linear fatty alcohols, glycerine, sorbitol, propylene glycol, fatty acid esters of monohydroxy or polyhydroxy alcohols, phthalates, hydrogenated castor oil, beeswax, acetylated monoglyceride, hydrogenated sperm oil, ethylenebis fatty acid esters, and higher fatty amides. The organic anti-blocking agents can be added in amounts that will fluctuate from 0.1 to 2% in weight.

Examples of anti-static agents are glycerine fatty acid, esters, sorbitan fatty acid esters, propylene glycol fatty acid esters, stearyl citrate, pentaerythritol fatty acid esters, polyglycerine fatty acid esters, and polyoxethylene glycerine fatty acid esters. An anti-static agent may range from 0.01 to 2% in weight. Lubricants may range from 0.1 to 2% in weight. A flame retardant will range from 0.01 to 2% in weight; ultra-violet light absorbers will range from 0.1 to 1 %; and antioxidants will range from 0.1 to 1 % in weight. The above compositions are expressed as percent of the total weight of the polymer blend.

Fillers, such as talc, silica, alumina, calcium carbonate, barium sulfate, metallic powder, glass spheres, barium stearate, calcium stearate, aluminum oxide, aluminum hydroxide, clay, titanium dioxide, diatomaceous earth and fiberglass, can be incorporated into the polymer composition in order to reduce cost or to add desired properties to the film or sheet. The amount of filler is desirably less than 10% of the total weight of the polymer composition as long as this amount does not alter the shrinking properties of the film or sheet when temperature is applied thereto.

The polymer composition for foamed sheets of the invention, can include impact modifiers. Examples of impact modifiers include high impact polystyrene (HIPS), styrene/butadiene block copolymers, styrene/ethylene/butene/styrene, block copolymers, styren/ethylene copolymers. The amount of impact modifier used is typically in the range of 0.5 to 25% of the total weight of polymer.

The foam is generally extruded at atmospheric pressure and as a result of the pressure release on the melt, the melt foams. The foam is cooled to ambient temperature typically below about 25° C., which is below the glass transition temperature of the polymer composition and the foam is stabilized.

In an embodiment of the invention, the foam can be extruded onto rollers as a relatively thick slab typically from about 1 to 3 inches thick. In this embodiment the foam density can vary from 2 to 15 lbs/ft³ (from about 0.03 to 0.24 g/cm³). The slab is cut into appropriate lengths (8 feet) and is generally used in the construction industry.

In another embodiment of the invention, thinner foamed sheets, typically from about 15 to about 300 mils thick can be extruded as slabs or as thin walled tubes, which are expanded and oriented over an expanding tubular mandrel to produce a foam tube, which is slit to produce sheet. These relatively thin sheets can be aged, typically 3 or 4 days and then can be thermoformed into articles, such as coffee cups, meat trays or “clam shells” or other containers suitable for use in heating food or liquids in a microwave oven.

Further to this embodiment, the foamed sheets can be at least 15, in some cases at least 20, in other cases at least 30, and in some instances at least 50 mils thick and can be up to 300, in some cases up to 250, in other cases up to 200, in some instance up to 150 and in other instances up to 125 mils thick. The thickness of the foamed sheet is determined by the intended end use and properties desired. The thickness of the foamed sheet can be any value or range between any of the values recited above.

More specifically, once the desired temperature is reached, the foamed sheet is formed into the desired shape by known processes such as plug assisted thermoforming where a plug pushes the foamed sheet into a mold of the desired shape. Air pressure and/or vacuum can also be employed to mold the desired shape.

In an embodiment of the invention, the thermoformed article is used for packaging food and one or more of the processes described above are carried out in a protected and/or sterile environment and/or atmosphere.

When used to package food or consumable liquids, the thermoformed article can be self closing or can include a container and a separate closure. Thus, in an embodiment of the invention, food or consumable liquids are placed into the container and the container is closed. Optionally, the container can then be shrink wrapped by a suitable material as is known in the art. Desirably, the shrink wrapping can include printing on its surface. Alternatively, a label, covering at least a portion of the container can be placed thereon.

In a particular embodiment of the invention, the label is placed in the thermoforming machine prior to forming the container and adheres to the formed container.

In an embodiment of the invention, the above-described foamed sheet has a foamed sheet flex modulus of at least 5,000 psi, in some cases at least 6,000 psi, in other cases at least 7,000 psi, in some instances at least 8,000 psi and in other instances at least 10,000 psi.

The foamed sheet flex modulus is determined using a standardized test coupon, which is subjected to three point bending under controlled conditions similar to those described in ASTM D-790 using an Instron Load Frame (4204 or 4400) with accessories, available from Instron Corporation, Canton, Mass. Load and deflection data are collected and evaluated. The slope of the load deflection curve, in the linear region, is a measure of the stiffness or rigidity of the material. Foam sheet materials, characteristically anisotropic, are evaluated in both the machine or “haul off” direction and the transverse or “across the sheet” direction. Flexural Stiffness is the initial linear behavior of the material when subjected to flexural deformation. Stiffness is quantified by the respective value of the slope of initial linear portion of the curve. Modulus is the slope of the load-deflection curve normalized to the thickness. The test conditions used are: (a) 1.5 inch span, (b) 1 inch per minute crosshead speed, (c) 4 inch (length) specimen.

In another embodiment of the invention, any of the foamed sheets described above can be coextruded or laminated with one or more materials to form a two-layer structure where the materials make up one layer (a cap layer) and the foamed sheet makes up the second layer or a sandwich structure foamed sheet, where the foamed sheet is included in the middle layer and the materials are included in the two outside layers. The materials that can be coextruded or laminated can be selected from crystal polystyrene, high impact polystyrenes, polyphenylene oxide, copolymers of styrene and maleic anhydride and/or C₁-C₁₂ linear, branched or cyclic alkyl (meth)acrylates, rubber-modified copolymers of styrene and maleic anhydride and/or C₁-C₁₂ linear, branched or cyclic alkyl (meth)acrylates, polycarbonates, polyamides (such as the nylons), polyesters (such as polyethylene terephthalate, PET), polyolefins (such as polyethylene, polypropylene, and ethylene-propylene copolymers), polyvinylidene fluoride, acrylonitrile/(meth)acrylate copolymers such as those available under the trade name BAREX® from BP Chemicals Inc., Cleveland, Ohio, ethylene/vinyl acetate copolymers, ethylene vinyl alcohol copolymers, and combinations thereof.

More particularly, the above-described method can include the step of extruding or laminating a solid sheet cap layer over at least a portion of a top surface of the foamed sheet.

Alternatively, the above-described method can include the steps of: extruding or laminating a top layer over at least a portion of a top surface of the foamed sheet and extruding or laminating a bottom layer over at least a portion of a bottom surface of the foamed sheet to form a sandwich structure foamed sheet.

As described above, the present invention provides articles that are formed by thermoforming any of the above-described foamed sheets to form articles. Because of the properties of the foamed sheets, the articles can include containers suitable for use in microwave heating of food.

The containers resulting from the present invention are suitable for packaging foods and can withstand the temperatures needed for heating foods in a microwave oven without the container breaking, deforming or leaking. Further, the containers maintain their form, especially upon removal of the container out of the microwave oven.

When the foamed sheet is coextruded as discussed above, the resulting multi-layer container is also suitable for use in microwave heating of food with the same type of desirable properties.

The present invention will further be described by reference to the following examples. The following examples are merely illustrative of the invention and are not intended to be limiting. Unless otherwise indicated, all percentages are by weight.

### EXAMPLES

In the Examples, the formed resins were injection molded into test specimens, which were tested by the following methods. The elongation at break was measured by ASTM-D638; the IZOD notched impact was measured by ASTM-D256; the VICAT heat distortion temperature was measured by ASTM-D1525; the Deflection Temperature Under Load (DTUL) was measured by ASTM-D648 on specimens annealed at 70° C. with 264 psi flexural stress; and the Instrumented Impact was measured by ASTM D-3763 with a 38 mm diameter hole clamp. The results are tabulated in the Tables below.

### Example 1

This example compares foamed sheets prepared from polystyrene with foamed sheets prepared according to the present invention. The polystyrene (sample A, comparative) had an Mn of 88,100, Mw of 339,300, Tg of 97.9° C., VICAT of 108° C. and was processed with 0.1wt. % zinc stearate.

The formulation (Sample B, inventive) for the foamed sheet according to the present invention was:

| | |
|---|---|
| Styrene | 68.4% |
| Maleic Anhydride | 8.0% |
| Polybutadiene rubber | 14.8% |
| SBS rubber | 4.5% |
| Polybutene H-100¹ | 4.0% |
| ANOX ™ PP18² | 0.3% |

¹INDOPOL ® H-100 available from AMOCO Chemical Company, Chicago, IL.
²Antioxidant available from Great Lakes Chemical Co., Indianapolis, IN.

A solution containing maleic anhydride, polybutene H-100, polybutadiene rubber, styrene-butadiene-styrene triblock copolymer (SBS) rubber, and ANOX™ PP18 (octadecycyl-3-(3′,5′-di-tert-butyl-4′-hydroxyphenyl)propionate) was dissolved in styrene monomer, and then fed continuously to a completely filled polymerization reactor equipped with a turbine agitator similar to that of U.S. Pat. No. 2,769,804. Benzoyl peroxide initiator, 0.01% of the main stream, was added into the reactor in a separate stream. The reactor was stirred so that it was well mixed. The reacting mass was maintained at 126° C. by cooling through the reactor jacket. The average residence time in the reactor was 2.7 hours. The exit stream contained 52% polymer and was then fed continuously into a devolatilizer in which the unreacted monomer was removed. The resultant resin contained 8% maleic anhydride, 14.8% polybutadiene rubber, 4.5% SBS rubber and 4% polybutene. Some of the polybutene was removed in the devolatilizing process. The final product was pelletized and molded into test specimens and testing was done using the methods outlined hereinabove.

The following results were obtained for the sample:

| | |
|---|---|
| Mw | 183,000 |
| Elongation at break | 12.7% |
| IZOD notched impact | 5.15 ft lbs/in |
| VICAT heat distortion temperature | 119.4° C. |

The polystyrene sample and rubber-modified styrene-maleic copolymer according to the invention were extruded using the following conditions. The blowing agent was 134a HFC used at about 5% with an extruded foam thickness of about 150 mils, a 4:1 blowup ratio and a nucleating package including a 50/50 mix of 3 μm ARTIC MIST® Talc (Luzenac, Inc., Oakville, Ontario) and SAFOAM® P MB available from Reedy International Corp., Keyport, N.J. The resin was fed at about 140 lbs/hr, nucleating agent at about 1 lb/hr, blowing agent at about 7 lbs/hr, the primary extruder temperatures were 370° F., 430° F., and 450° F. and the secondary extruder temperatures were 212° F., 255° F. and 265° F.

Properties of the resulting sheets are shown in the following table.

| | Sample A | | | |
|---|---|---|---|---|
| | (comparative) | | Sample B | |
| Basis Weight | 21 | g/Ft² | 18 | g/Ft² |
| Sheet Thickness | 0.11 | in | 0.08 | in |
| Number of cells across sheet | 25 | | 18 | |
| Cell size | 0.11 | mm | 0.11 | mm |
| Cells/inch | 227 | | 225 | |
| foamed sheet flex modulus | 18,000 | psi | 13,970 | psi |
| (machine direction) | | | | |
| foamed sheet flex modulus | 22.680 | psi | 9,640 | psi |
| (transverse direction) | | | | |

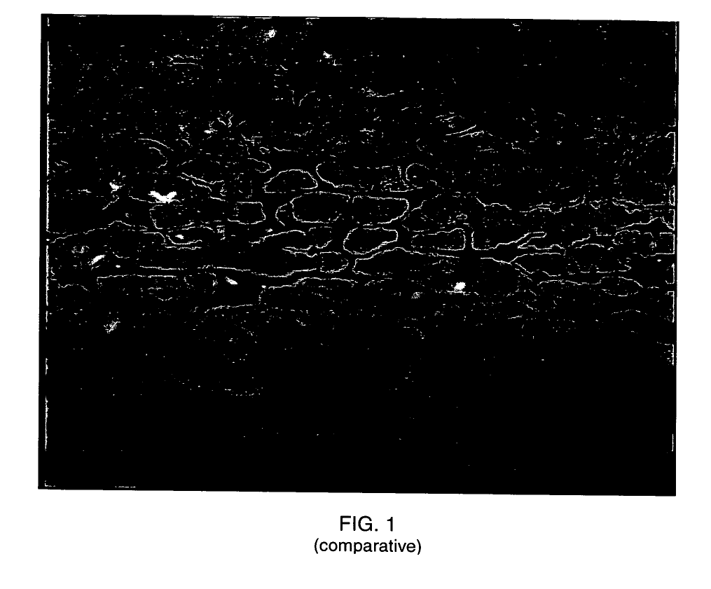

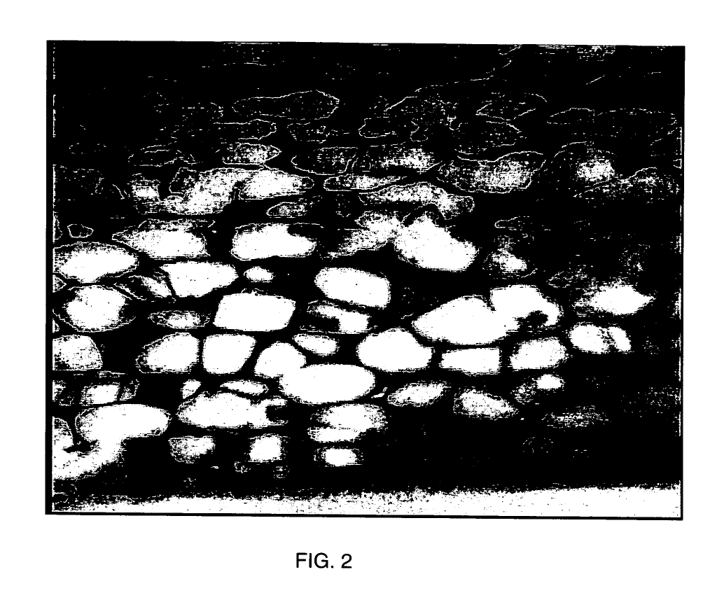

Containers were thermoformed from samples A and B and evaluated for environmental stress crack resistance (ESCR) by placing the listed items in the container and microwaving the contents on the high setting of a 600-watt microwave oven for the period of time indicated. A pass indicates that no hole was made in the container. A fail indicates that a hole formed and material leaked out of the container. Photo Micrographs of a cross section of the foam sheet of sample A (FIG. 1) and sample B (FIG. 2) show foam structure of the samples. Results are shown in the table below.

| | | Sample A | |
|---|---|---|---|
| | Time (seconds) | (Comparative) | Sample B |
| Duck Sauce | 30 | Pass | Pass |
| Soy Sauce | 30 | Fail | Pass |
| Hot Chili Sauce | 30 | Fail | Pass |
| Mayonnaise | 10 | Fail | Pass |
| Saltine Crackers | 30 | Pass | Pass |

The results demonstrate that foamed sheets made according to the invention have physical properties competitive with polystyrene but containers made from the inventive foamed sheets have superior properties as containers for use in microwave ovens.

### Example 2

A solution containing 4.2% maleic anhydride, 1.6% polybutene H-100, 7.5% butadiene rubber, and 0.16% ANOX™ PP18 was dissolved in styrene monomer, and then fed continuously to a completely filled polymerization reactor equipped with a turbine agitator similar to that of U.S. Pat. No. 2,769,804. Benzoyl peroxide initiator, 0.01% of the main stream, was added into the reactor in a separate stream. The reactor was stirred so that it was well mixed. The reacting mass was maintained at 126° C. by cooling through the reactor jacket. The average residence time in the reactor was 2.7 hours. The exit stream contained 52% polymer and was then fed continuously into a devolatilizer in which the unreacted monomer was removed. The resultant resin contained 8% maleic anhydride, 15% butadiene rubber and 2.5% polybutene. Some of the polybutene was removed in the devolatilizing process. The final product was pelletized.

The pellets can be extruded using the following conditions. The blowing agent is n-pentane used at about 5% with an extruded foam thickness of about 150 mils, a 4:1 blowup ratio and a nucleating package including a 50/50 mix of 3μm ARTIC MIST® Talc and SAFOAM® P MB available from Reedy International Corp., Keyport, N.J. The resin is fed at about 140 lbs/hr, nucleating agent at about 1 lb/hr, blowing agent at about 7 lbs/hr, the primary extruder temperatures are 370° F., 430° F., and 450° F. and the secondary extruder temperatures is 212° F., 255° F. and 265° F. A foamed sheet is produced that is thermoformed into containers for use in microwave ovens.

### Example 3-7

A solution containing maleic anhydride, low molecular weight polybutadiene (or polybutene), and styrene-butadiene rubber was dissolved in styrene monomer as indicated in the table below, and then fed continuously to a completely filled polymerization reactor equipped with a turbine agitator similar to that of U.S. Pat. No. 2,769,804. t-butyl peroctoate (t-butyl peroxy-2-ethylhexanoate), 120 ppm based on the main stream, was added into the reactor in a separate stream. The reactor was stirred so that it was well mixed. The reacting mass was maintained at 126° C. by cooling through the reactor jacket. The average residence time in the reactor was 2.7 hours. The exit stream contained 52% polymer and was then fed continuously into a devolatilizer in which the unreacted monomer was removed. The final products were pelletized.

| | Exam- | Exam- | | | |
|---|---|---|---|---|---|
| | ple 3 | ple 4 | Example 5 | Example 6 | Example 7 |
| Styrene (wt. %) | 85 | 84 | 85 | 84 | 84.5 |
| Maleic | 5 | 5 | 5 | 5 | 5 |
| Anhydr. (wt. %) | | | | | |
| Styr-butadiene | 9 | 9 | 9 | 9 | 9 |
| rubber (wt. %) | | | | | |
| Polybutadiene | 2100 | 2100 | 5000 | 5000 | — |
| (Mn) | | | | | |
| Polybutadiene | 1 | 2 | 1 | 2 | 0 |
| (wt. %) | | | | | |
| Polybutene | — | — | — | — | 1.5 |
| (Mn = 910) | | | | | |
| (wt. %) | | | | | |

The following results were obtained for the samples:

| | Ex. 3 | Ex. 4 | Ex. 5 | Ex. 6 | Ex. 7 |
|---|---|---|---|---|---|
| Elongation at break (%) | 21.5 | 14.8 | 20.6 | 23.2 | 25.9 |
| IZOD notched impact (lbs/in) | 2.7 | 3.7 | 3.2 | 4.0 | 3.3 |
| VICAT temperature (° C.) | 122.2 | 119.4 | 121.4 | 120.8 | 119.2 |

The pellets can be extruded using the following conditions. The blowing agent is n-pentane used at about 5% with an extruded foam thickness of about 150 mils, a 4:1 blowup ratio and a nucleating package including a 50/50 mix of 3 μm ARTIC MIST® Talc and SAFOAM® P MB available from Reedy International Corp., Keyport, N.J. The resin is fed at about 140 lbs/hr, nucleating agent at about 1 lb/hr, blowing agent at about 7 lbs/hr, the primary extruder temperatures are 370° F., 430° F., and 450° F. and the secondary extruder temperatures is 212° F., 255° F. and 265° F. A foamed sheet is produced that is thermoformed into containers for use in microwave ovens.

### Examples 8-12

The rubber-modified styrene-maleic copolymer (Example B) according to the invention from Example 1 was extruded using the following conditions. Decomposition of SAFOAM® FP-40 (citric acid/bicarbonate) acted as the blowing agent at the levels indicated in the table below. The sheet had an extruded foam thickness of about 150 mils, a 4:1 blowup ratio and a talc nucleating agent was used at the levels indicated in the table below was used. The resin was fed at about 140 lbs/hr, nucleating agent at about 1 lb/hr, blowing agent at about 7 lbs/hr, the primary extruder temperatures were 475° F., 495° F., 505° F. and 520° F. and the secondary extruder temperatures were 212° F., 255° F. and 265° F. Properties of the resulting sheet are shown in the table below.

| | Ex. 8 | | | | |
|---|---|---|---|---|---|
| | (comparative) | Ex. 9 | Ex. 10 | Ex. 11 | Ex. 12 |
| Copolymer of Ex. 1 | 100 | 98.5 | 98 | 97.5 | 98 |
| (wt. %) | | | | | |
| Talc | — | 1 | 1 | 1 | 1 |
| SAFOAM ® | — | 0.5 | 1 | 1.5 | 1 |
| FP-40 | | | | | |
| Sheet | 0.02 | 0.02 | 0.02 | 0.02 | 0.02 |
| Thickness (in.) | | | | | |
| Basis | 55.6 | 50.0 | 52.8 | 47.2 | 50.4 |
| Weight (g/ft.) | | | | | |

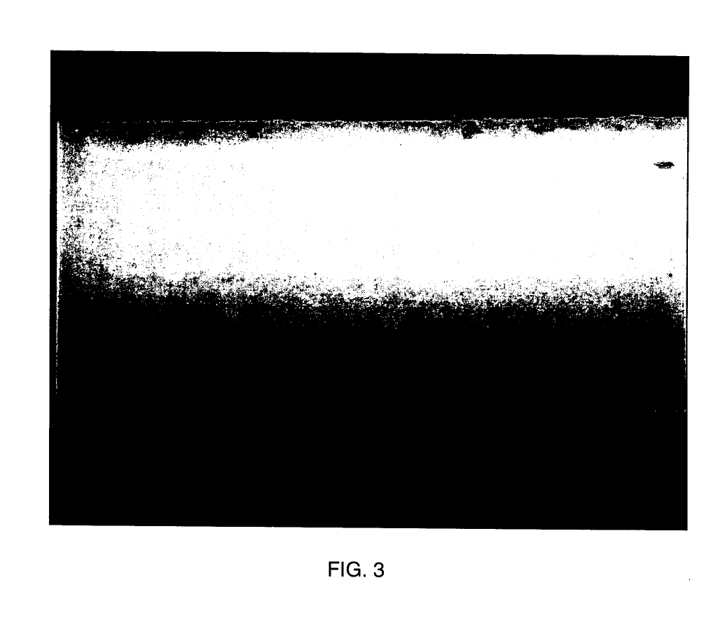

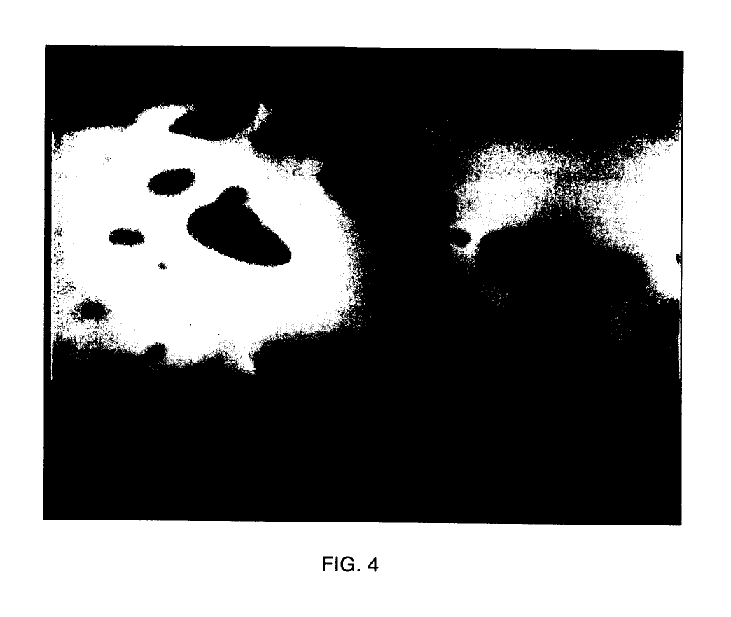

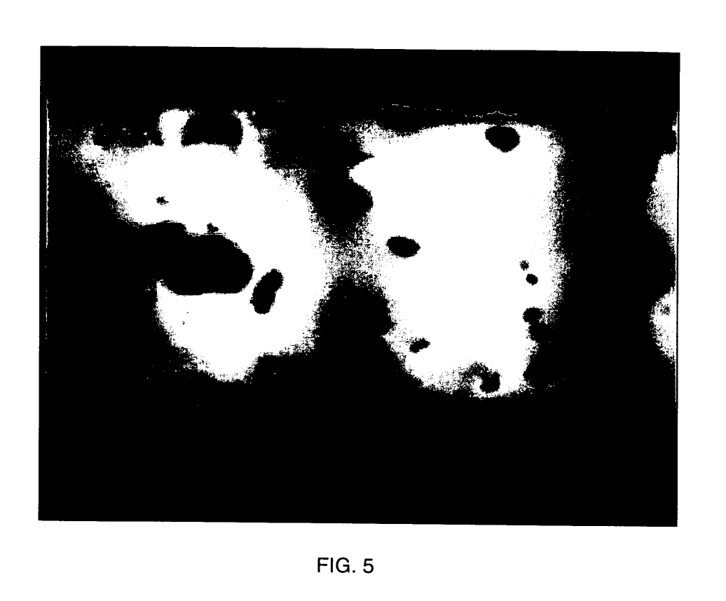

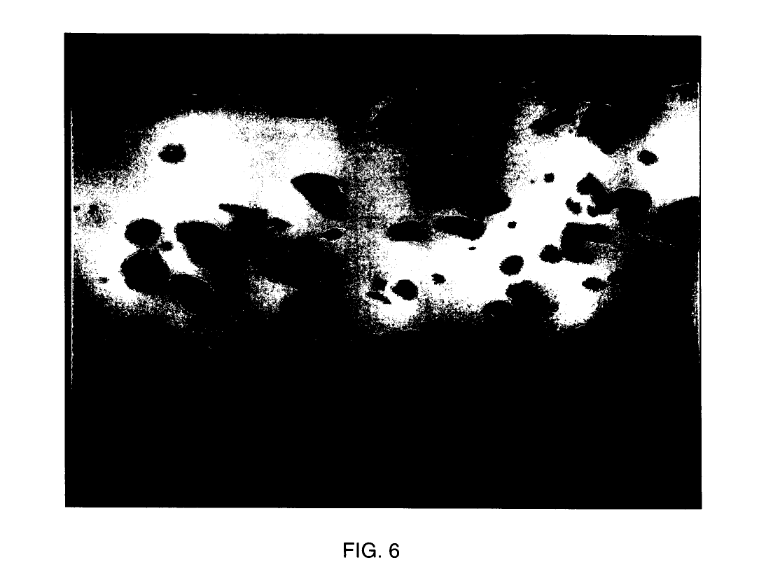

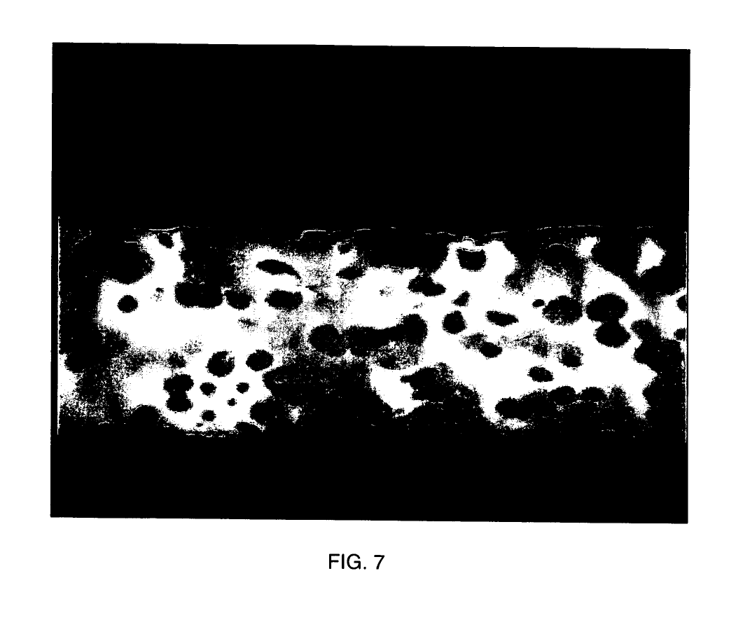

Photomicrographs of a cross-section of the foamed sheet produced in Example 8 (FIG. 3), Example 9 (FIG. 4), Example 10 (FIG. 5), Example 11 (FIG. 6) and Example 12 (FIG. 7). Generally, the inclusion of nucleating agents results in a larger cellular structure in the foamed sheet and a lower basis weight fro the sheet.

The foamed sheets can be thermoformed to form containers for use in microwave ovens.

### Example 13

This example demonstrates the formation of a foamed sheet according to the present invention using the following formulation:

| | |
|---|---|
| Styrene | 67.2% |
| Maleic Anhydride | 8.5% |
| Polybutadiene rubber | 15.4% |
| SBS rubber | 4.6% |
| Polybutene H-100¹ | 4.0% |
| ANOX ™ PP18² | 0.3% |

¹INDOPOL ® H-100 available from AMOCO Chemical Company, Chicago, IL.
²Antioxidant available from Great Lakes Chemical Co., Indianapolis, IN.

A solution containing maleic anhydride, polybutene H-100, polybutadiene rubber, styrene-butadiene-styrene triblock copolymer (SBS) rubber, and ANOX™ PP18 (octadecycyl-3-(3′,5′-di-tert-butyl-4′-hydroxyphenyl)propionate) was dissolved in styrene monomer, and then fed continuously to a completely filled polymerization reactor equipped with a turbine agitator similar to that of U.S. Pat. No. 2,769,804. Benzoyl peroxide initiator, 0.01% of the main stream, was added into the reactor in a separate stream. The reactor was stirred so that it was well mixed. The reacting mass was maintained at 126° C. by cooling through the reactor jacket. The average residence time in the reactor was 2.7 hours. The exit stream contained 52% polymer and was then fed continuously into a devolatilizer in which the unreacted monomer was removed. The resultant resin contained 8.5% maleic anhydride, 15.4% polybutadiene rubber, 4.6% SBS rubber and 4% polybutene. The final product was pelletized and molded into test specimens and testing was done using the methods outlined hereinabove. The pellets contained about 2,150 ppm styrene monomer.

The following results were obtained for the sample:

| | |
|---|---|
| Mw | 164,000 |
| Rubber particle size (vol. in μm) | 2.4 |
| Elongation at break | 7.7% |
| IZOD notched impact | 5.06 ft lbs/in |
| VICAT heat distortion temperature | 122.3° C. |

The polystyrene sample and rubber-modified styrene-maleic copolymer according to the invention were extruded using the following conditions. The blowing agent was 134a HFC used at about 5% with an extruded foam thickness of about 150 mils, a 4:1 blowup ratio and a nucleating package including a 50/50 mix of 3 μm ARTIC MIST® Talc (Luzenac, Inc., Oakville, Ontario) and SAFOAM® P MB available from Reedy International Corp., Keyport, N.J. The resin was fed at about 140 lbs/hr, nucleating agent at about 1 lb/hr, blowing agent at about 7 lbs/hr, the primary extruder temperatures were 370° F., 430° F., and 450° F. and the secondary extruder temperatures were 212° F., 255° F. and 265° F. The foamed sheet contained 1,779 ppm styrene monomer (a reduction of 370 ppm).

The foamed sheets can be thermoformed to form containers for use in microwave ovens.

### Examples 14-16

These examples demonstrate combining RSMA (the rubber-modified styrene-maleic copolymer (Example B) according to the invention from Example 1) with a CSMA (crystal styrene-maleic copolymer (DYLARK® 232 available from NOVA Chemicals Inc., Pittsburgh, Pa.)) to prepare foamed sheets.

Weight ratios of RSMA and CSMA as indicated in the table below and 1.1 % talc based on the combined weight of the RSMA and CSMA were fed to an extruder. The blowing agent was 134a HFC used at about 5% with an extruded foam thickness of about 90-100 mil. The primary extruder temperatures were set at 400° F., 480° F., and 450° F. and the secondary extruder temperatures were set at 260° F. and 270° F. The foamed sheet had properties as indicated in the table below.

| | Example 13 | | Example 14 | | Example 15 | |
|---|---|---|---|---|---|---|
| RSMA | 100% | | 75% | | 25% | |
| CSMA | — | | 25% | | 75% | |
| Talc | 1.1% | | 1.1% | | 1.1% | |
| Basis Weight | 24 | g/Ft² | 24 | g/Ft² | 24 | g/Ft² |
| Sheet Thickness (mils) | 90-95 | | 90-100 | | 85-92 | |
| Cells/inch | 200 | | 204 | | 211 | |
| foamed sheet flex | 14,700 | psi | 18,500 | psi | 24,100 | psi |
| modulus | | | | | | |
| (machine direction) | | | | | | |
| foamed sheet flex | 18,000 | psi | 16,700 | psi | 28,600 | psi |
| modulus | | | | | | |
| (transverse direction) | | | | | | |

The data demonstrate the excellent foamed sheet properties obtained when the present rubber-modified styrene-maleic copolymer is used alone or in combination with unmodified styrene-maleic copolymer to prepare foamed sheets according to the invention.

### Example 17

This example addresses preparing a sheet that includes a solid sheet cap layer over a foamed sheet according to the invention. Rubber modified SMA resin pellets prepared according to Example 1 of U.S. patent Application Publication 2005/0020756 A1 are added to an extruder (Welex, Inc.). An extruded (unfoamed, opaque) sheet is produced, approximately 3 to 6 mils thick, by operating the extruder using temperature zones of 400°, 425°, and 475° C. A foamed sheet prepared according to Example 1, sample A is fed below the extruded sheet and the extruded and foamed sheets are fed between rolls which results in the extruded sheet sticking to the foam sheet. The resulting extrusion coated sheet has an opaque unfoamed layer and a foamed sheet layer.

The present invention has been described with reference to specific details of particular embodiments thereof. It is not intended that such details be regarded as limitations upon the scope of the invention except insofar as and to the extent that they are included in the accompanying claims.

1. A foamed sheet comprising a polymer composition containing a copolymer formed by polymerizing a mixture comprising:
about 40% to about 90% by weight of one or more styrenic monomers; and
about 5% to about 45% by weight of one or more maleate-type monomers; and combining the copolymer with
about 0.1% to about 25% by weight of one or more elastomeric polymers having a number average molecular weight of greater than 6,000; and
about 0.1% to about 10% by weight of one or more low molecular weight polymers comprising one or more monomers according to the formula CH₂═CR³R², wherein R³ is H or a C₁-C₃ alkyl group and R² is a C₁-C₂₂ linear, branched or cyclic alkyl or alkenyl group, wherein the low molecular weight polymer has a number average molecular weight of from 400 to 6,000 and can optionally include one or more functional groups selected from the group consisting of hydroxyl, amine, epoxy, carboxylic acid, carboxylic acid esters, and carboxylic acid anhydrides.

2. The foamed sheet according to claim 1, wherein the styrenic monomers are selected from the group consisting of styrene, p-methyl styrene, α-methyl styrene, tertiary butyl styrene, dimethyl styrene, nuclear brominated or chlorinated derivatives thereof and combinations thereof.

3. The foamed sheet according to claim 1, wherein the maleate-type monomers are selected from the group consisting of maleic anhydride, maleic acid, fumaric acid, C₁-C₁₂ linear, branched or cyclic alkyl esters of maleic acid, C₁-C₁₂ linear, branched or cyclic alkyl esters of fumaric acid, itaconic acid, C₁-C₁₂ linear, branched or cyclic alkyl esters of itaconic acid, and itaconic anhydride.

4. The foamed sheet according to claim 1, wherein the elastomeric polymers are selected from the group consisting of homopolymers of butadiene or isoprene, and random, block, AB diblock, or ABA triblock copolymers of a conjugated diene with a styrenic monomer and/or acrylonitrile.

5. The foamed sheet according to claim 1, wherein the elastomeric polymers are one or more block copolymers selected from the group consisting of diblock and triblock copolymers of styrene-butadiene, styrene-butadiene-styrene, styrene-isoprene, styrene-isoprene-styrene, partially hydrogenated styrene-isoprene-styrene and combinations thereof.

6. The foamed sheet according to claim 1, wherein the low molecular weight polymers comprise repeat units resulting from the polymerization of one or monomers selected from the group consisting of 1-butene, isobutylene, 2-butene, isoprene, butadiene, diisobutylene, 1-pentene, 2-pentene, 1-hexene, 2-hexene, 3-hexene, 1,3-hexadiene, 2,4-hexadiene, isoprenol, ethylene, propylene and combinations thereof.

7. The foamed sheet according to claim 1, wherein the low molecular weight polymers contain one or more functional groups selected from the group consisting of hydroxyl, amine, epoxy, carboxylic acid, C₁-C₁₂ linear, branched or cyclic alkyl carboxylic acid esters, and carboxylic acid anhydride.

8. The foamed sheet according to claim 1, wherein the styrenic and maleate-type monomers and copolymers formed therefrom comprise a continuous phase and the elastomeric polymers comprise a dispersed particulate phase having particles with an average particle size of from about 0.1 microns to about 11 microns.

9. The foamed sheet according to claim 8, wherein the aspect ratio of the dispersed particles is from about 1 to about 5.

10. The foamed sheet according to claim 1, wherein the weight average molecular weight of the formed copolymer is from about 20,000 to about 1,000,000.

11. The foamed sheet according to claim 1, wherein the polymer composition has a VICAT softening temperature of greater than 100° C.

12. The foamed sheet according to claim 1 having a foamed sheet flex modulus of at least 5,000 psi.

13. The foamed sheet according to claim 1 further comprising one or more other polymers blended with the polymer composition.

14. The foamed sheet according to claim 13, wherein the other polymers are selected from the group consisting of crystal polystyrene, high impact polystyrenes, polyphenylene oxide, copolymers of styrene and maleic anhydride and/or C₁-C₁₂ linear, branched or cyclic alkyl (meth)acrylates, rubber-modified copolymers of styrene and maleic anhydride and/or C₁-C₁₂ linear, branched or cyclic alkyl (meth)acrylates, polycarbonates, polyamides, polyesters, polyolefins, polyvinylidene fluoride, acrylonitrile/(meth)acrylate copolymers, ethylene/vinyl acetate copolymers, ethylene vinyl alcohol copolymers, and combinations thereof.

15. The foamed sheet according to claim 13, wherein the polymer composition is present at from 10% to 90% and the other polymers are present at from 10% to 90% of the blend based on the weight of the blend.

16. The foamed sheet according to claim 1 further comprising one or more additives selected from the group consisting of heat stabilizers, light stabilizers, softening agents; plasticizers, dyes, pigments; anti-blocking agents; slip agents; lubricants; coloring agents; antioxidants; ultraviolet light absorbers; fillers; anti-static agents; impact modifiers, and combinations thereof.

17. An article produced from the foamed sheet according to claim 1.

18. The article according to claim 17, wherein the article is produced by thermoforming the foamed sheet.

19. A container suitable for use in microwave heating of food formed from the foamed sheet composition according to claim 1.

20. A method of making a foamed sheet comprising:
providing a polymer composition in polymer melt form prepared by polymerizing a mixture comprising:
about 40% to about 90% by weight of one or more styrenic monomers; and about 5% to about 45% by weight of one or more maleate-type monomers to form a copolymer; and combining the copolymer with
about 0.1% to about 25% by weight of one or more elastomeric polymers having a number average molecular weight of greater than 6,000; and
about 0.1% to about 10% by weight of a low molecular weight polymer comprising one or more monomers according to the formula CH₂═CR³R², wherein R¹ is H or a C₁-C₃ alkyl group and R² is a C₂-C₂₂ linear, branched or cyclic alkyl or alkenyl group, wherein the low molecular weight polymer has a number average molecular weight of from 400 to 6,000 and can optionally include one or more functional groups selected from the group consisting of hydroxyl, amine, epoxy, carboxylic acid, carboxylic acid esters, and carboxylic acid anhydrides;
injecting into the polymer melt composition from 2 to 15 weight % based on the polymer composition of one or more blowing agents;
mixing the blowing agent with the polymer composition; and
extruding the mixture of blowing agent and polymer composition to provide a foamed sheet.

21-37. (canceled)

38. The method of making a foamed sheet according to claim 20, wherein prior to the step of extruding the mixture of blowing agent and polymer composition, a step including blending one or more other polymers with the polymer composition is performed.

39-45. (canceled)

46. The method of making a foamed sheet according to claim 20 comprising the step of extruding or laminating a solid sheet cap layer over at least a portion of a top surface of the foamed sheet.

47-51. (canceled)

52. The method of making a foamed sheet according to claim 20 comprising the steps of:
extruding or laminating a top layer over at least a portion of a top surface of the foamed sheet: and
extruding or laminating a bottom layer over at least a portion of a bottom surface of the foamed sheet to form a sandwich structure foamed sheet.

53. (canceled)

54. A sandwich structure foamed sheet made according to the method of claim 52.

55-57. (canceled)

58. A foamed sheet made according to the method of claim 38.

59-67. (canceled)

68. The method of making a foamed sheet according to claim 38 comprising the steps of:
extruding or laminating a top layer over at least a portion of a top surface of the foamed sheet: and
extruding or laminating a bottom layer over at least a portion of a bottom surface of the foamed sheet to form a sandwich structure foamed sheet.

69. (canceled)

70. A sandwich structure foamed sheet made according to the method of claim 68.

71-73. (canceled)

74. A two layer foamed sheet comprising a first layer that includes the foamed sheet according to claim 1 and a second layer comprising one or more resin selected from the group consisting of crystal polystyrene, high impact polystyrenes, polyphenylene oxide, copolymers of styrene and maleic anhydride and/or C₁-C₁₂ linear, branched or cyclic alkyl (meth)acrylates, rubber-modified copolymers of styrene and maleic anhydride and/or C₁-C₁₂ linear, branched or cyclic alkyl (meth)acrylates, polycarbonates, polyamides, polyesters, polyolefins, polyvinylidene fluoride, acrylonitrile/(meth)acrylate copolymers, ethylene/vinyl acetate copolymers, ethylene vinyl alcohol copolymers, and combinations thereof.

75-76. (canceled)

77. A sandwich structure foamed sheet comprising a middle layer that includes the foamed sheet according to claim 1, and a top layer and a bottom layer independently comprising a resin selected from the group consisting of crystal polystyrene, high impact polystyrenes, polyphenylene oxide, copolymers of styrene and maleic anhydride and/or C₁-C₁₂ linear, branched or cyclic alkyl (meth)acrylates, rubber-modified copolymers of styrene and maleic anhydride and/or C₁-C₁₂ linear, branched or cyclic alkyl (meth)acrylates, polycarbonates, polyamides, polyesters, polyolefins, polyvinylidene fluoride, acrylonitrile/(meth)acrylate copolymers, ethylene/vinyl acetate copolymers, ethylene vinyl alcohol copolymers, and combinations thereof.

78-79. (canceled)

80. A container suitable for use in microwave heating of food formed by thermoforming a foamed sheet comprising a polymer composition containing a copolymer formed by polymerizing a mixture comprising:
about 40% to about 90% by weight of one or more styrenic monomers; and
about 5% to about 45% by weight of one or more maleate-type monomers; and combining the copolymer with
about 0.1% to about 25% by weight of one or more elastomeric polymers having a number average molecular weight of greater than 6,000; and
optionally up to about 10% by weight of one or more low molecular weight polymers comprising one or more monomers according to the formula CH₂═CR³R², wherein R³ is H or a C₁-C₃ alkyl group and R² is a C₁-C₂₂ linear, branched or cyclic alkyl or alkenyl group, wherein the low molecular weight polymer has a number average molecular weight of from 400 to 6,000 and can optionally include one or more functional groups selected from the group consisting of hydroxyl, amine, epoxy, carboxylic acid, carboxylic acid esters, and carboxylic acid anhydrides.
